(19) United States
(12) Patent Application Publication
(10) Pub. No.: US 2011/0123640 A1
(43) Pub. Date: May 26, 2011

(54) NOVEL FERMENTED MILK PRODUCT AND USE THEREOF

(75) Inventors: Yoshie Yabumoto, Kanagawa (JP); Hisanori Tani, Tokyo (JP); Kazuto Suzuki, Tokyo (JP); Tetsumi Yabumoto, Kanagawa (JP); Yuriko Yoshida, Kanagawa (JP)

(73) Assignee: Kabushiki Kaisha Fiss, Yokohama-shi, Kanagawa (JP)

(21) Appl. No.: 12/996,917

(22) PCT Filed: Jun. 8, 2009
(86) PCT No.: PCT/JP2009/060439

§ 371 Date: Dec. 8, 2010

(30) Foreign Application Priority Data
Jun. 10, 2008 (JP) 2008-151310

(51) Int. Cl. A61K 35/20; C12N 1/20; A61P 1/00; A61P 31/00; A23C 9/127

(52) U.S. Cl. 424/535; 435/252.9; 426/580

(57) ABSTRACT

Disclosed is a fermented milk product, which is useful for the promotion of good health. The fermented milk product is produced by fermenting a milk raw material with a combination of microorganisms consisting of (1) Lactobacillus kefiri and (2) at least one microorganism selected from the group consisting of Kazachstania turicensis, Kazachstania unispora and Kluyveromyces marxianus. Preferably, the fermented milk product is produced by fermenting the milk raw material with a combination of microorganisms consisting of at least Lactobacillus kefiri strain P-IF, Lactobacillus kefiri strain P-B1, Kazachstania turicensis strain P-Y3, Kazachstania unispora strain P-Y4 and Kluyveromyces marxianus strain P-Y5.

### TECHNICAL FIELD

The present invention relates to a fermented milk product obtained by fermenting a milk raw material with a combination of microorganisms comprising lactic acid bacteria and yeasts, an antimicrobial composition comprising the fermented milk product as an active ingredient, and a food or drink comprising the fermented milk product.

### BACKGROUND ART

It is known that yogurt and the like, which is a lactic-acid-bacterium fermentation product, contains antimicrobials such as bacteriocins. And it is thought that, with the antimicrobial properties, the growth of enterobacteria that badly affect intestinal environment is inhibited, while the growth of enterobacteria that give good influence on the intestinal environment is promoted.

Regarding antimicrobial properties over enterobacteria with a lactic acid bacterium, it is described in the following patent document 1 that a lactic acid producing bacterium such as Lactobacillus kefiri was administered to an animal to inhibit the incidence rate and proliferation of E. coli O157:H7 and other pathogenic bacteria.

Patent document 1: JP 2006-501311 A

### DISCLOSURE OF THE INVENTION

### Problems to be Solved by the Invention

However the above-mentioned patent document 1 was described as an art for inhibition of generation of pathogenic bacteria in ruminants and was not necessarily the art for useful for health promotion inhuman such as amelioration of the intestinal environment.

Therefore, the objective of the present invention is to provide a fermented milk product which has a high antimicrobial activity over enterobacteria that badly affect intestinal environment in human and is useful for health promotion such as amelioration of the intestinal environment.

### Means for Solving the Problems

The present invention provides as follows.

[1] A fermented milk product characterized in that the product is obtained by fermenting a milk raw material with a combination of microorganisms comprising (1) Lactobacillus kefiri and (2) at least one microorganism selected from the group consisting of Kazachstania turicensis, Kazachstania unispora, and Kluyveromyces marxianus.
[2] A fermented milk product according to the above [1], wherein the Lactobacillus kefiri is Lactobacillus kefiri strain P-IF (FERM BP-10896).
[3] A fermented milk product according to the above [1] or [2], wherein the product is obtained by fermenting the milk raw material with a combination of microorganisms comprising at least Lactobacillus kefiri strain P-IF (FERM BP-10896), Lactobacillus kefiri strain P-B1 (FERM BP-11115), Kazachstania turicensis strain P-Y3 (FERM BP-11116), Kazachstania unispora strain P-Y4 (FERM BP-11117), and Kluyveromyces marxianus strain P-Y5 (FERM BP-11118).
[4] An antimicrobial composition comprising the fermented milk product above mentioned as an active ingredient.
[5] A composition for amelioration of the intestinal environment comprising the fermented milk product above mentioned as an active ingredient.
[6] A food or drink comprising the fermented milk product above mentioned.

### Effects of the Invention

The fermented milk product according to the present invention inhibits the growth of enterobacteria that badly affect intestinal environment. Therefore, health promotion such as amelioration of the intestinal environment would be achieved by oral intake thereof. And also the antimicrobial composition according to the present invention, with the antimicrobial properties, inhibits the growth of enterobacteria that badly affect intestinal environment, while promotes the growth of enterobacteria that give good influence on the intestinal environment. And also the food or drink according to the present invention provides a food or drink useful for health promotion such as amelioration of the intestinal environment.

### BRIEF DESCRIPTION OF THE DRAWINGS

FIG. 1 shows an electron microscope picture of the “strain P-IF”.

### BEST MODE FOR CARRYING OUT THE INVENTION

The microorganisms used in the present invention are Lactobacillus kefiri of lactic acid bacterium, Kazachstania turicensis of yeast, Kazachstania unispora of yeast, and Kluyveromyces marxianus of yeast, and preferably are Lactobacillus kefiri strain P-IF (FERM BP-10896, National Institute of Advanced Industrial Science and Technology, International Patent Organism Depositary, Tsukuba Central 6, 1-1-1 Higashi, Tsukuba, Ibaraki, Japan) (hereinafter also called “strain P-IF”)), Lactobacillus kefiri strain P-B1 (FERM BP-11115, National Institute of Advanced Industrial Science and Technology, International Patent Organism Depositary, Tsukuba Central 6, 1-1-1 Higashi, Tsukuba, Ibaraki, Japan) (hereinafter also called “strain P-B1”)), Kazachstania turicensis strain P-Y3 (FERM BP-11116, National Institute of Advanced Industrial Science and Technology, International Patent Organism Depositary, Tsukuba Central 6, 1-1-1 Higashi, Tsukuba, Ibaraki, Japan) (hereinafter also called “strain P-Y3”)), Kazachstania unispora strain P-Y4 (FERMBP-11117, National Institute of Advanced Industrial Science and Technology, International Patent Organism Depositary, Tsukuba Central 6, 1-1-1 Higashi, Tsukuba, Ibaraki, Japan) (hereinafter also called “strain P-Y4”)), and Kluyveromyces marxianus strain P-Y5 (FERM BP-11118, National Institute of Advanced Industrial Science and Technology, International Patent Organism Depositary, Tsukuba Central 6, 1-1-1 Higashi, Tsukuba, Ibaraki, Japan) (hereinafter also called “strain P-Y5”)).

Each of “strain P-IF”, “strain P-B1”, “strain P-Y3”, “strain P-Y4”, and “strain P-Y5” was isolated by picking up from single colony after culturing on an agar media respectively, and their DNA sequences were determined individually to identify each species. Precisely, with regard to “strain P-IF” and “strain P-B1”, their 16S rDNA nucleotide sequences, that are the ribosomal RNA gene on genome DNA, were determined and then analyzed though homology search. And with regard to “strain P-Y3”, “strain P-Y4” and “strain P-Y5”, their 26S rDNA-D1/D2 nucleotide sequences, that are the ribosomal RNA gene on genome DNA, were determined and then analyzed through homology search. Consequently the “strain P-IF” was estimated to be a member of the Lactobacilllus kefiri, because it was revealed that the nucleotide sequence of the “strain P-IF” showed a similarity of 99.6% to that of Lactobacillus kefiri. Similarly the “strain P-B1” was estimated to be a member of the Lactobacilllus kefiri, because it was revealed that the nucleotide sequence of the “strain P-B1” showed a similarity of 99.6% to that of Lactobacillus kefiri. And the “strain P-Y3” was estimated to be a member of the Kazachstania turicensis, because it was revealed that the nucleotide sequence of the “strain P-Y3” showed a similarity of 100% to that of Kazachstania turicensis. And the “strain P-Y4” was estimated to be a member of the Kazachstania unispora, because it was revealed that the nucleotide sequence of the “strain P-Y4” showed a similarity of 100% to that of Kazachstania unispora. And furthermore the “strain P-Y5” was estimated to be a member of the Kluyveromyces marxianus, because it was revealed that the nucleotide sequence of the “strain P-Y5” showed a similarity of 100% to that of Kluyveromyces marxianus.

The microbial characteristics of the “strain P-IF” are described in detail as follows.

In table 1, the microbial characteristics of the “strain P-IF” are shown.

TABLE 1

| Testing items | | “strain P-IF” |
|---|---|---|
| Culture temperature (° C.) | | 30 |
| Cell shape | | Rod |
| | | (0.8-1.0 × 1.5-2.0 μm) |
| Gram staining | | + |
| Spore forming | | − |
| Motility | | − |
| Colony appearance | | Medium: MRS agar |
| | | Culture time: 48 hrs |
| | | Diameter: 2.0-3.0 mm |
| | | Color: milky |
| | | Shape: circular |
| | | Upheaval: convex |
| | | Periphery: wrinkle |
| | | Surface: smooth |
| | | Transparency: opaque |
| | | Viscosity: butter-like |
| Growth temperature | 37 | + |
| test (° C.) | 45 | − |

| | |
|---|---|
| Catalase activity | − |
| Oxidase activity | − |
| Acid/gas production from | +/− |
| glucose | |
| (Acid production/gas | |
| production) | |
| O/F test | +/+ |
| (Oxidative/Fermentative) | |

In table 2, also the sugar utilization characteristics of the “strain P-IF” are shown.

TABLE 2

| Sugar | Assimi- | Sugar | Assimi- | Sugar | Assimi- |
|---|---|---|---|---|---|
| substrate | lation | substrate | lation | substrate | lation |
| Control | − | inositol | − | melicitose | − |
| glycerol | − | mannitol | − | raffinose | − |
| erythritol | − | sorbitol | − | starch | − |
| D-arabinose | − | α-methyl-D- | − | glycogen | − |
| | | mannoside | | | |
| L-arabinose | + | α-methyl-D- | − | xylitol | − |
| | | glucoside | | | |
| ribose | + | N-acetyl- | − | gentiobiose | − |
| | | glucosamine | | | |
| D-xylose | − | amygdalin | − | D-turanose | − |
| L-xylose | − | arbutin | − | D-lyxose | − |
| adonitol | − | esculin | − | D-tagatose | − |
| β-methyl-D- | − | salicin | − | D-fucose | − |
| xyloside | | | | | |
| galactose | + | cellobiose | − | L-fucose | − |
| glucose | + | maltose | + | D-arabitol | − |
| fructose | + | lactose | + | L-arabitol | − |
| mannose | − | melibiose | + | gluconate | + |
| sorbose | − | saccharose | − | 2-keto- | − |
| | | | | gluconate | |
| rhamnose | − | trehalose | − | 5-keto- | − |
| | | | | gluconate | |
| dulcitol | − | inulin | − | | |

The “strain P-IF” was estimated to be a member of the Lactobacilllus kefiri, because it was revealed that the 16S rDNA nucleotide sequence of the “strain P-IF”, which was determined and then analyzed through homology search utilizing with microbial 16S rDNA sequence databases, showed a similarity of 99.6% to that of Lactobacillus kefiri. And the above-mentioned microbial characteristics and sugar utilization characteristics were corresponded to the characteristics of Lactobacillus kefiri.

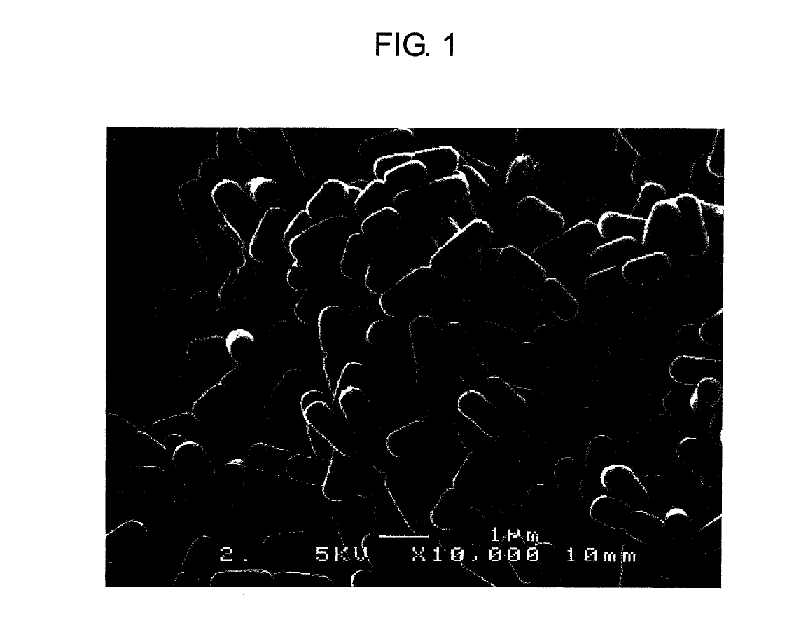

However, it was different from traditional Lactobacillus kefiri in utilization of galactose as a carbon source. And, when it was cultured on liquid media in a test tube, carbonic acid gas was produced only by a slight vibration like champagne. This was also characteristics never observed in traditional Lactobacillus kefiri. Furthermore, when observed under an electron microscope as shown in FIG. 1, the “strain P-IF” has such characteristics as being freely adhered to other cells and developing proliferation in a manner of three-dimensional cell division. Meanwhile, ordinary strains of L. kefiri develop proliferation in a manner of lengthwise-dimensional cell division. This is suggestive to some difference in the structure of carbohydrate chain on the microbial surface.

It was also revealed that, when the “strain P-IF” was cultured and fermented in MRS liquid media, the pH of those media reached very low level of pH4.3, but the “strain P-IF” could continue to grow under that severe condition. This is suggestive to the capability of the “strain P-IF” to have resistance over stomach acid to get to an intestinal tract as being alive.

As described above, it was understandable that, although it belongs to a microbial member of Lactobacillus kefiri, the “strain P-IF” is a novel type of strain having morphologically unique characteristics.

As for the microbial characteristics of the “strain P-B1”, “strain P-Y3”, “strain P-Y4”, and “strain P-Y5”, they have similar microbial characteristics to those of the same microbial species, respectively.

The isolated strains of “strain P-IF”, “strain P-B1”, “strain P-Y3”, “strain P-Y4”, and “strain P-Y5” can be cultured in accordance with the method known in public, for example in case of “strain P-IF” and “strain P-B1”, these lactic acid bacteria can be cultured using MRS media “Lactobacilli MRS Broth” (product name, Difco) in an anaerobic and static culture. And also in case of the “strain P-Y3”, “strain P-Y4″and “strain P-Y5”, these yeasts can be cultured for example using YM media “glucose 1%, peptone 0.5%, malt extract 0.3%, yeast extract 0.3%, agar 2%” (product name, Difco) in an anaerobic and static culture.

The fermented milk product of the present invention is obtained by fermenting a milk raw material with a combination of microorganisms comprising (1) Lactobacillus kefiri and (2) at least one microorganism selected from the group consisting of Kazachstania turicensis, Kazachstania unispora, and Kluyveromyces marxianus. As the Lactobacillus kefiri, Lactobacillus kefiri strain P-IF (FERN BP-10896) may be used preferably.

Further, it is more preferable that the fermented milk product is obtained by fermenting the milk raw material with a combination of microorganisms comprising at least Lactobacillus kefiri strain P-IF (FERN BP-10896), Lactobacillus kefiri strain P-B1 (FERN BP-11115), Kazachstania turicensis strain P-Y3 (FERN BP-11116), Kazachstania unispora strain P-Y4 (FERN BP-11117), and Kluyveromyces marxianus strain P-Y5 (FERN BP-11118).

As the milk raw material, for example, milk, whey, fermented milk, lactic acid bacterium drink, skim milk, powdered skim milk, prepared powdered milk, powdered milk, concentrated milk, concentrated skim milk, condensed milk, condensed skim milk, sweetened condensed milk, sweetened condensed skim milk, etc. may be used.

Fermentation of the milk raw material for the present invention may be carried out in accordance with such a procedure in which the milk raw material is inoculated with a starter comprising the above-mentioned combination of microorganisms and exposed to fermenting and culturing conditions known in public. It is preferable that the milk raw material is fermented at first at 24-26° C. for 20-28 hrs, and then at 20-22° C. for 20-28 hrs, since then they are aged at a temperature dropped to 10-12° C. for 42-54 hrs. Alternatively, there is other culturing procedure, in which an initial culture is started at 24-26° C., and then the culture temperature is dropped to 20-22° C. linearly, and an additional fermentation is carried out at 20-22° C. for 20-28 hrs, and then the culture is aged at 10-12° C. for 42-54 hrs. According to such procedure, it becomes easier to produce active ingredients, because microorganisms individually contained in the above-mentioned combination of microorganisms are cultured symbiotically.

Fermented milk product, deeply fermented with such procedure as mentioned above, may be used for the present invention with or without sterilization. Alternatively, the supernatant, which is derived from the culture after separation and removing of cell bodies with a centrifugation or a filtration, may be also used for the fermented milk product according to the present invention, while the present invention is not restricted to such embodiments.

When the fermented milk product according to the present invention is taken orally, a daily amount of intake (as a solid content) is preferably from 0.01 (mg/kg of body weight) to 10 (g/kg of body weight), and more preferably from 0.05 (mg/kg of body weight) to 1 (g/kg of body weight), while there is no particular limitation.

The fermented milk product according to the present invention may be used in various fields such as pharmaceuticals, health foods, processed foods etc. And there is no particular limitation in the form, so the fermented milk product may be used in a form of preparation such as liquid, syrup, jelly, capsule, powder, tablet, granule, troche drug, as those can be prepared by methods known in public appropriately. Furthermore, the fermented milk product according to the present invention may be also used as an ingredient in various foods or drinks.

The fermented milk product according to the present invention, with its antimicrobial properties shown below in the examples, is useful as an active ingredient in an antimicrobial composition. And it may be particularly useful as an active ingredient in a composition for amelioration of the intestinal environment improvement, since it showed strong antimicrobial activity over anaerobic microflora of enterobacteria, including the members of genus Clostridium generally called villain bacteria.

### EXAMPLES

Hereinafter, the present invention is described precisely with examples, but these examples do not limit the scope of the present invention.

### Production Example 1

### Preparation of a Fermented Milk Product

The milk raw material (whey), which was inoculated with a combination of microorganisms as a starter containing “strain P-IF”, “strain P-B1”, “strain P-Y3”, “strain P-Y4”, and “strain P-Y5”, was fermented first at 25° C. for 24 hrs and then at 21° C. for 24 hrs, and since then aged at a temperature dropped to 11° C. for 48 hrs.

### Example 1

### Antimicrobial Activity Test

An antimicrobial activity of the fermented milk product obtained in Example 1 was investigated. Precisely, we added the same volume of n-butanol to the fermented milk product (1 L) from which cell bodies were removed beforehand by a filtration, and mixed them completely and made them stand overnight. Then the butanol layer was separated from the water layer by a centrifugation, and both layers were collected respectively. Subsequently, the butanol layer was concentrated and dried under a decompression condition. The dried resultant was dissolved in a small amount of deionized water and filtered by a glass filter, then dialyzed it against deionized water with a dialysis tube of fractioning molecular weight of 1 kDa. The dialyzed external solution, low molecular weight fraction, was collected and concentrated under a decompression condition and freeze-dried.

Freeze-dried resultant of n-butanol extracted-low molecular weight fraction obtained was dissolved at a concentration of 10 mgl/ml in deionized water, and adjusted to pH 4.5 by 1/10N lactic acid, and the solution by 25 or 50 μl was absorbed in a paper disc (8 mm, ADVANTEC).

Meanwhile, a part of human feces was sampled and diluted with sterilized physiological saline, and then cultured anaerobically using GAM bouillon (NISSUI PHARMACEUTICAL CO., LTD) and equipped with a simple jar for anaerobic culture called as “Anaeropackkennki” (product name, Mitubishi Gas Chemical Company, Inc.). After 24 hrs, a small amount of the culture was picked up onto a laboratory dish, and added thereto a plate count agar media (Merk & Co., Inc.), and made a plate in a manner of mixing and dilution.

Each of the paper discs that absorbed the solution of every sample was put onto the agar flat plate media including microbes derived from feces, and cultured at 35° C. for 24 hrs, and then kept to observe the inhibitory zone around the paper disc. The results are show in Table

### Comparative Example 1

Milk was inoculated with a powdered kefir A (a commercial product: household type to be added as a kefir seed to milk for fermentation to eat) and fermented at 25° C. for 18 hrs. The same volume of n-butanol was added to the fermented liquor, and prepared in the same manner as Example 1 the freeze-dried resultant of n-butanol extracted-low molecular weight fraction, and carried out an antimicrobial activity test. The results are show in Table 3.

### Comparative Example 2

The same volume of n-butanol was added to the lactic acid bacterium beverage B (a commercial product), and prepared in the same manner as Example 1 the freeze-dried resultant of n-butanol extracted-low molecular weight fraction, and carried out an antimicrobial activity test. The results are show in Table 3.

### Comparative Example 3

The same volume of n-butanol was added to the yogurt C (a commercial product), and prepared in the same manner as Example 1 the freeze-dried resultant of n-butanol extracted-low molecular weight fraction, and carried out an antimicrobial activity test. The results are show in Table 3.

TABLE 3

| | Degree of inhibitory zone | | | |
|---|---|---|---|---|
| Sample | n-butanol-low molecular weight fraction | | | |
| Example 1 | ++(25 | μl) | +++(50 | μl) |
| Comparative | −(25 | μl) | −(50 | μl) |
| Example 1 | | | | |

| Comparative | −(50 μl) |
|---|---|
| Example 2 | |
| Comparative | −(50 μl) |
| Example 3 | |

The numerical values within each parentheses exhibit the volume absorbed by discs.
−: negative
+: weakly positive (clear-cut inhibitory zone having)
++: positive (clear-cut inhibitory zone having φ below 10 mm including φ8 mm of disc)
+++: strongly positive (clear-cut inhibitory zone having φ over 10 mm including φ8 mm of disc)

As shown in Table 3, the fermented milk product of the present invention was observed to have strong antimicrobial activity (Example 1). On the other hand, the commercial fermented milk products did not show any formation of inhibitory zone, so antimicrobial activity was not observed (Comparative Example 1-3). Further, although the result is not shown in herewith, it is notable that an obtained culture which was obtained as follows; that is, “strain P-IF” was inoculated in a MRS liquid media, and made proliferated for 48 to 72 hrs at 25° C. to 10¹° to 10¹¹ cfu/ml, then a part of the cultured media inoculated in 3 ml of skim milk media (prepared by adding 10 weight parts of skim milk powder to 90 weight parts of ion-exchanged water, and mixing them, and sterilizing them at 121° C. for 15 min), and made fermented for 5 days at 25° C. under a static and anaerobic condition; was observed to have some antimicrobial activity. Therefore, the fermentation of milk raw material by “strain P-IF” was indicated to be a main factor for expression of the above-mentioned antimicrobial activity.

The microbes submitted to this test were derived from the feces and anaerobic microflora of enterobacteria, including the members of genus Clostridium generally called villain bacteria. As the fermented milk product for the invention showed strong antimicrobial activity over those microflora, the fermented milk product was indicated to have more superior activity for amelioration of the intestinal environment improvement.

### DEPOSIT NUMBER

(1) FERM BP-10896: Lactobacillus strain kefiri P-IF
(2) FERM BP-11115: Lactobacillus strain kefiri P-B1
(3) FERM BP-11116: Kazachstania strain turicensis P-Y3
(4) FERM BP-11117: Kazachstania strain unispora P-Y4
(5) FERM BP-11118: Kluyveromyces strain marxianus P-Y5

1. A fermented milk product characterized in that the product is obtained by fermenting a milk raw material with a combination of microorganisms comprising (1) Lactobacillus kefiri strain P-IF (FERM BP-10896) and (2) at least one microorganism selected from the group consisting of Kazachstania turicensis, Kazachstania unispora, and Kluyveromyces marxianus.

2. (canceled)

3. A fermented milk product according to claim 1, wherein the product is obtained by fermenting the milk raw material with a combination of microorganisms comprising at least Lactobacillus kefiri strain P-IF (FERM BP-10896), Lactobacillus kefiri strain P-B1 (FERM BP-11115), Kazachstania turicensis strain P-Y3 (FERM BP-11116), Kazachstania unispora strain P-Y4 (FERM BP-11117), and Kluyveromyces marxianus strain P-Y5 (FERM BP-11118).

4. An antimicrobial composition comprising the fermented milk product according to claim 1 as an active ingredient.

5. A composition for amelioration of the intestinal environment comprising the fermented milk product according to claim 1 as an active ingredient.

6. A food or drink comprising the fermented milk product according to claim 1.

7. An antimicrobial composition comprising the fermented milk product according to claim 2 as an active ingredient.

8. A composition for amelioration of the intestinal environment comprising the fermented milk product according to claim 2 as an active ingredient.

9. A food or drink comprising the fermented milk product according to claim 2.

10. A strain of Lactobacillus kefiri P-IF (FERM BP-10896).
